(19) United States
(12) Patent Application Publication
(10) Pub. No.: US 2016/0223527 A1
(43) Pub. Date: Aug. 4, 2016

(54) An Immunological Assay To Detect And Quantify Acetylamantadine

(71) Applicant: BIOMARK TECHNOLOGIES INC, Richmond (CA)

(72) Inventor: Brian Cheng, Chesterfield, MO (US)

(73) Assignee: BioMark Technologies Inc., Richmond, BC (CA)

(21) Appl. No.: 14/915,616

(22) PCT Filed: Aug. 29, 2014
(86) PCT No.: PCT/CA2014/050833

§ 371 Date: Feb. 29, 2016

Related U.S. Application Data
(60) Provisional application No. 61/871,642, filed on Aug. 29, 2013.

(51) Int. Cl. G01N 33/53

(52) U.S. Cl. CPC G01N 33/5308

(57) ABSTRACT

An immunological assay detects and quantifies acetylamantadine in the urine or other bodily fluids of a person who has taken a small dose of amantadine. The quantification of acetylamantadine can be used to quantify SSAT activity and elevated SSAT activity is an indication of diseases including, but not limited, to inflammations and cancers.

### BACKGROUND OF THE INVENTION

1. Field of the Invention

The present invention relates to methods and compositions for the quantification of spermine/spermidine N¹-acetyltransferase (SSAT) enzymatic activity.

2. Description of the Related Art

Spermidine/spermine N¹-acetyltransferase (SSAT) is ubiquitously distributed in mammalian tissues and plays a role in catabolism and elimination of polyamines from cells. SSAT is an inducible enzyme that catalyzes the transfer of an acetyl group from an acetyl-coenzyme A to the aminopropyl moiety of the polyamines. This action by SSAT facilitates polyamine degradation, excretion, and cycling and/or intracellular cycling. In this manner SSAT participates in the maintenance of polyamine homeostasis in mammalian cells. However, in normal or uninduced mammalian tissues SSAT is present at very low levels.

Induction of SSAT expression can be caused by different drugs, growth factors, polyamines, polyamine analogues, toxic substances, hormones and physiological stimuli. Although all of the aforementioned compounds could cause induction of SSAT expression, induction occurs at different times for each individual compound. The regulation of SSAT expression occurs at the levels of transcription, mRNA stability, mRNA translation and protein stability. Induction or over-expression of SSAT is usually required for there to be sufficient SSAT enzyme present in cells or 100,000×g supernatant before in-vitro experiments can be successfully undertaken.

While current literature teaches that SSAT is an acetylating enzyme specifically for substrates including spermine and spermidine or its analogues, SSAT activity, SSAT enzyme kinetics and assay methodology for non-spermine/spermidine substrates of SSAT have not been understood. Current methods exist to quantify SSAT activity. However, these techniques are dependent on highly skilled personnel and complicated experimental methods. More specifically, there has been a need for assay methodology which quantifies the activity of SSAT through detection of acetylated forms of non-spermine. Spermidine substrates of SSAT, including amantadine, may be used to detect various pathological conditions.

Traditional methods such as Gas Liquid Chromatography (GLC), High Pressure Liquid Chromatography (HPLC) alone or coupled with mass spectroscopy are being used for assaying acetylated metabolite such as acetylamantadine. The detection sensitivity requires parts per billion of the target analyte which becomes a challenge. GLC and HPLC have been shown to be effective for in-vitro assays but may not be practical for in-vivo assays. Employment of deuterated analyte as an internal standard for HPLC-MS-MS method to assay acetylamantadine was successful.

These traditional methods require labor intensive analytical service resulting in high operating costs and capital costs. All of these add to the inefficiencies of the healthcare economy. In addition, biological samples must be logistically handled and shipped to a centralized laboratory for assay. These operations may result in sample quality changes and could result in false interpretation of the result.

### SUMMARY OF THE INVENTION

It is an object of the present invention to provide an immunological assay that detects, and preferably quantifies, acetylamantadine in urine or other bodily fluids of a mammal or patient given a small dose of amantadine.

It is disclosed that it is possible to inhibit, with a sample of urine containing acetylamantadine, the binding of anti-acetylamantadine polyclonal antibodies to acetylamantadine bound to a substrate. Polyclonal antibodies that specifically recognize acetylamantadine are generated by immunizing warm-blooded animals with an immunogen acetylamantadine cross-linked to a carrier protein or a universal T cell epitope preferably with an adjuvant. The methods of cross-linking proteins, universal T cell epitopes, and adjuvants are conventional. The polyclonal antibodies that specifically recognize acetylamantadine are absorbed with amantadine because there are high concentrations of amantadine in the urine of a mammal or patient who has taken a small dose of amantadine. This can be done by conventional methods, such as putting the antibodies on a column of agarose beads covalently coupled with amantadine and taking the flow-through from the column. Also provided is a quantitative assay with a standard curve with measured amounts of acetylamantadine.

The form of in-vitro testing diagnostic (IVD) based on this disclosure is an immunological assay that detects, and may quantify, acetylamantadine at the clinical office. This may allow for quick medical decisions and may indirectly measure SSAT activity in the body. A test methodology at the point of care is therefore desirable to minimize sample instability and reduce healthcare costs. The form of IVD at the clinical office may allow quick medical decisions.

There may be designs of IVD based on this disclosure of an immunological assay that measures acetyl amantadine in the urine of a mammal who has taken a small dose of amantadine.

The polyclonal antibodies specific for acetylamantadine which are absorbed by amantadine may be used to detect or quantify acetylamantadine in the presence of an excess of amantadine.

The polyclonal antibodies specific for acetylamantadine which are absorbed by amantadine may be used to detect or quantify spermine/spermidine N¹-acetyltransferase (SSAT) activity in the body.

### BRIEF DESCRIPTIONS OF DRAWINGS

The invention will be more readily understood from the following description of the embodiments thereof given, by way of example only, with reference to the accompanying drawings, in which:

FIG. 1 is a graph illustrating polyclonal affinity-purified antibodies against acetylamantadine absorbed with amantadine inhibited by small concentrations of acetylamantadine in a standard curve;

FIG. 2 is a graph illustrating polyclonal affinity-purified antibodies against acetylamantadine absorbed with amantadine inhibited by small concentrations of acetylamantadine and large concentrations of amantadine;

FIG. 3 is a graph illustrating polyclonal affinity-purified polyclonal antibodies against acetylamantadine inhibited by small concentrations of acetylamantadine and large concentrations of amantadine;

FIG. 4 is a graph illustrating serum from a rabbit inhibited by small concentrations of acetylamantadine and large concentrations of amantadine; and

FIG. 5 is a graph illustrating the results of an acetylamandine competitive ELISA with signal reading against acetylamantadine competitive concentration.

### BRIEF DESCRIPTION OF THE PREFERRED EMBODIMENTS

Disclosed herein is an immunological assay that may be used to detect or quantify acetylamantadine in a mammal who has taken a small dose of amantadine.

A bodily fluid sample is taken from the mammal and incubated with a known concentration of polyclonal antibodies specific for acetylamantadine. The acetylamantadine in the sample will inhibit the polyclonal antibodies specific for acetylamantadine from binding to acetylamantadine attached to a substrate. There are many ways to attach acetylamantadine to the substrate where the antibody signal is detected. Biotinylated acetylamantadine was attached to streptavidin attached to the substrate in the example disclosed herein. The substrate is where the signal is detected when anti-acetylamantadine antibodies bind to acetylamantadine if the anti-acetylamantadine antibodies are not inhibited by acetylamantadine in the sample. If the concentration of acetylamantadine in the sample is exceeding the threshold of abnormal concentrations, the signal on the substrate may be completely inhibited. It would be understood by a person skilled in the art that many designs of in-vitro testing diagnostic (IVD) that detects acetylamantadine in a sample may be used. For example, coloured particles, like gold particles, are bound to acetylamantadine and abnormal concentrations of acetylamantadine in the sample inhibit the binding of coloured particles to a test line bound with anti-acetylamantadine antibodies.

### Preparation of Acetylamantadine Protein Conjugates

A conjugate of a carrier protein ovalbumin is linked by 1-ethyl-3-(3-dimethylaminopropyl)-carbodiimide (EDC) to an amine-derivative of acetylamantadine using the manufacturer's instructions. In this example, the manufacturer was Thermo Fisher Scientific Inc. of 3747 N Meridian Rd, Rockford, Ill., United States of America, 61101. Another immunogen that rabbits were immunized with was a conjugate of keyhole-limpet hemocyanin linked by formaldhyde to an amine-derivative of acetylamantadine. Another immunogen that rabbits were immunized with was a conjugate of the biotin-binding protein avidin coupled to an excess of biotinylated 4-amino-1-N-acetylamantadine. The biotinylation was carried out using standard methods and instructions from the manufacturer which, in this example, was Thermo Fisher Scientific Inc. of 3747 N Meridian Rd, Rockford, Ill., United States of America, 61101.

The amine-derivative of acetylamantadine was synthesized as described in the following synthetic scheme.

### Synthesis of Adamantylamine Derivatives

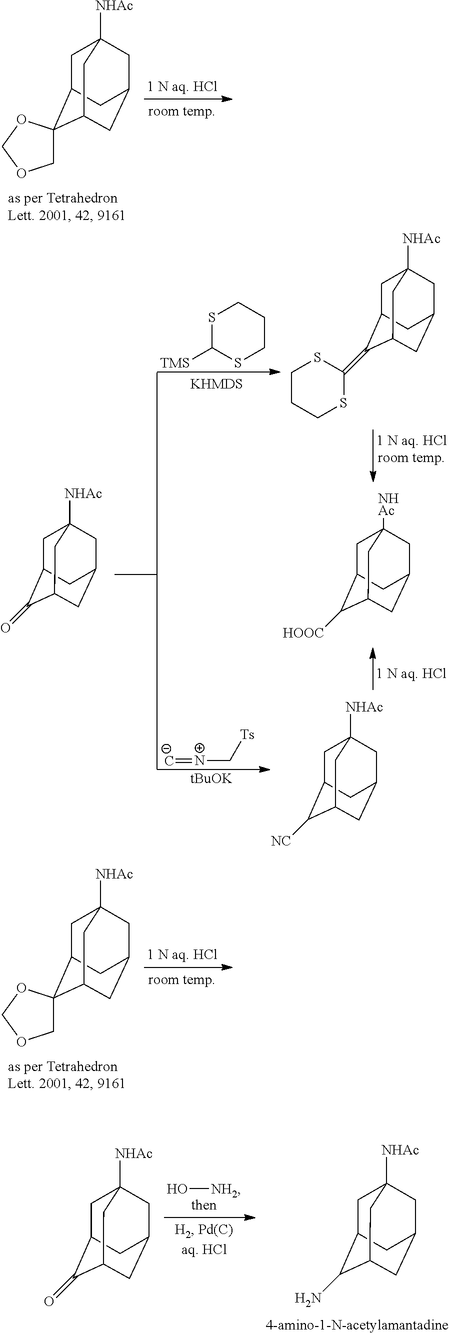

Immunization of rabbit:

A New Zealand white rabbit was immunized intramuscularly with 250 μg of the acetylamantadine-ovalbumin conjugate emulsified in complete Freund's adjuvant. The rabbit was boosted multiple times with acetylamantadine-ovalbumin conjugate, twice with 100 μg of acetylamantadine-conjugate keyhole-limpet hemocyanin and once with 4 pg of avidin coupled with an excess of biotinylated acetylamantadine with alum as an adjuvant. The rabbit was boosted with 100 μg of the acetylamantadine-ovalbumin conjugate 14 days before the rabbit was euthanized and bled out.

### Preparation of Immunoglobin G (IgG):

The rabbit blood was allowed to clot and the sera were collected. The IgG was collected using beads coated with Protein A and the IgG was eluted from the column by pH 2 glycine buffer and neutralized to pH 7.2.

Preparation of columns:

Cyanogen-bromide coupled agarose beads were conjugated with either an amine derivative of acetylamantadine or an amine derivative of amantadine.

Affinity-purified antibodies against acetylamantadine:

The IgG was flowed over a column of cyanogen-bromide coupled agarose beads conjugated with acetylamantadine and the antibodies against acetylamantadine bound to the column. The column was washed extensively with phosphate buffered saline. The affinity-purified antibodies against acetylamantadine were eluted from the column by pH 2 glycine buffer and neutralized to pH 7.2.

Affinity-purified antibodies against acetylamantadine absorbed with amantadine:

The affinity-purified antibodies against acetylamantadine were flowed over a column of cyanogen-bromide coupled agarose beads conjugated with amantadine and the peak of IgG was taken.

Quantifying acetylamantadine with affinity-purified antibodies against acetylamantadine absorbed with amantadine:

Enzyme-linked immunosorbent assay (ELISA) plates were coated with the biotin-binding protein streptavidin at 2 μg/mL and blocked with skim milk. The ELISA plates were then washed with phosphate-buffered saline with an automated washer. Biotinylated acetylamantadine at 2 ng/mL was added to the streptavidin-coated ELISA plates so that acetylamantadine was bound to the streptavidin. The ELISA plates were then washed to remove the free biotinylated acetylamantadine. The soluble acetylamantadine or the amatidine was titrated in a separate 96-well plate and incubated for an hour with a small, previously determined concentration of anti-acetylamantadine affinity-purified antibodies absorbed with amantadine that gives a clear signal in the ELISA plates coated with acetylamantadine. The smallest amount of affinity-purified antibodies against acetylamantadine absorbed with amantadine gives the greatest sensitivity for acetylamantadine. Then the titrations of acetylamantadine and amantadine incubated for one hour with a constant amount of anti-acetylamantadine affinity-purified antibodies absorbed with amantadine were added to the ELISA plates coated with acetylamantadine.

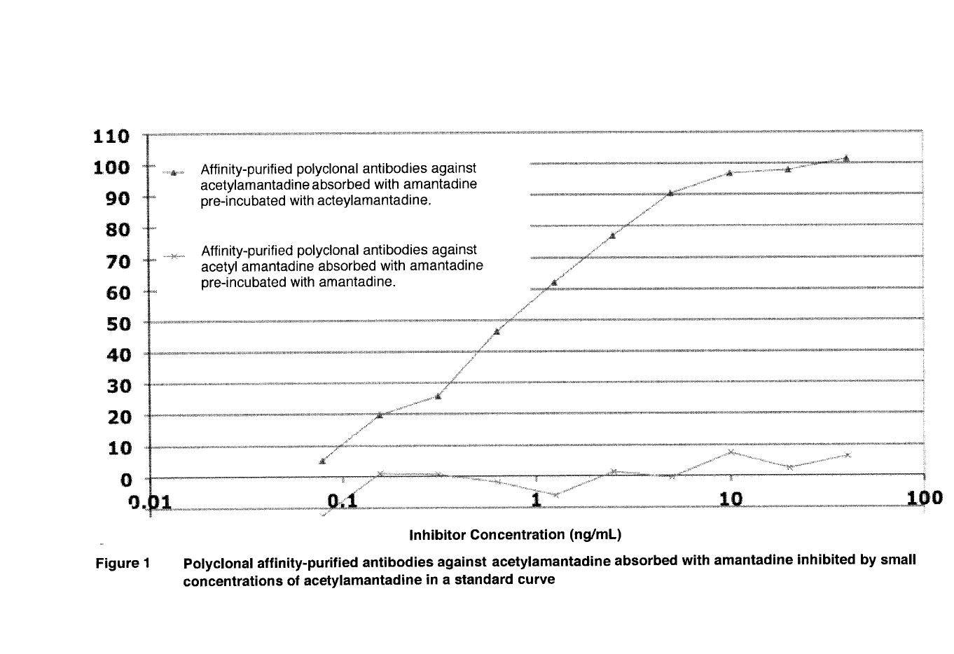

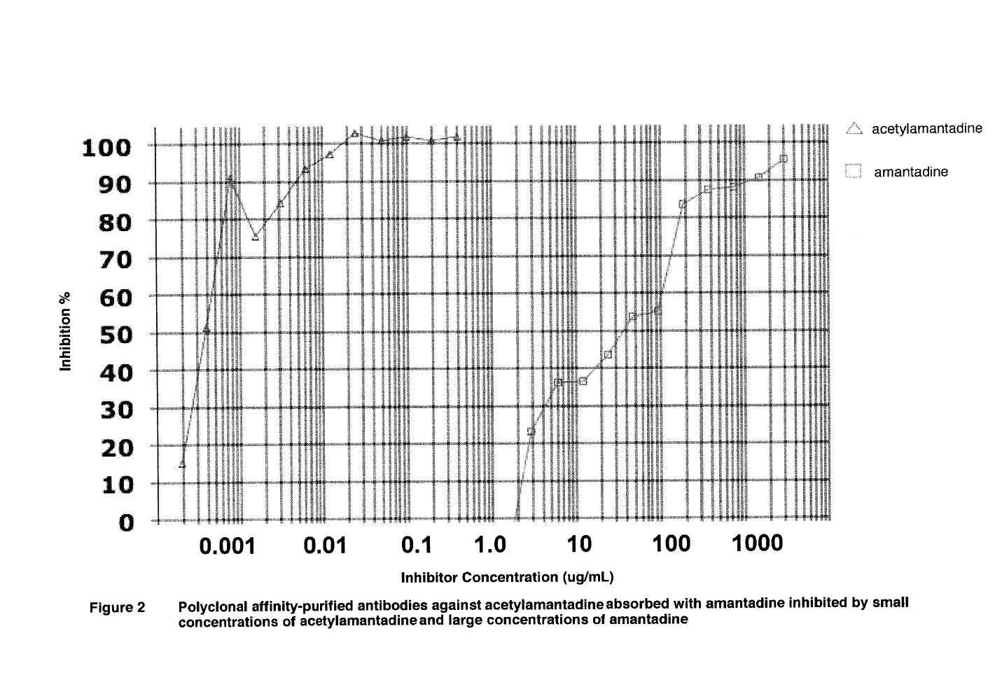

The ELISA was incubated for two hours and then the ELISA was developed by adding goat antibodies against rabbit IgG conjugated with alakine phosphatase. The substrate was added and the ELISA developed by standard methods and the optical densities were assayed by an ELISA reader and are shown in FIG. 1. In particular, FIG. 1 shows a standard curve showing inhibition from 100 pg/mL from 10 ng/mL. If one makes quadruplicate assays of samples of urine, one can accurately quantify acetylamantadine in the assay. If the urine sample inhibits 100%, one can make a dilution of the sample and accurately quantify acetylamantadine in the assay. There was significant inhibition (50%) of the binding in the ELISA with ˜400 pg/mL of soluble acetylamantadine added to the anti-acetylamantadine affinity-purified antibodies absorbed with amantadine as shown in FIG. 2. In contrast, there was significant inhibition (50%) with only 40 μg/mL of amantadine as shown in FIG. 2.

Quantifying acetylamantadine with affinity-purified antibodies against acetylamantadine:

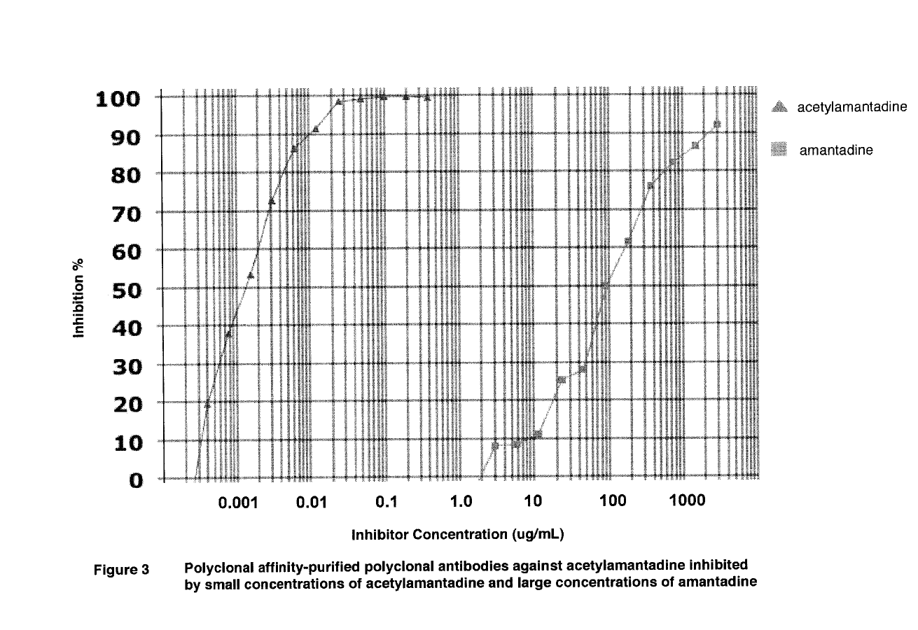

The affinity-purified antibodies from this individual rabbit that bound to acetylamantadine, and not absorbed with amantadine, also worked in this immunological assay as shown in FIG. 3. There was significant inhibition (50%) of the binding in the ELISA with ˜1.3 ng/ mL of soluble acetylamantadine added to the anti-acetylamantadine affinity-purified antibodies absorbed with amantadine as shown in FIG. 2. In contrast there was significant inhibition (50%) with only 100 μg/mL of amantadine as shown in FIG. 2.

Quantifying acetylamantadine with serum of a rabbit immunized repeatedly against acetylamantadine:

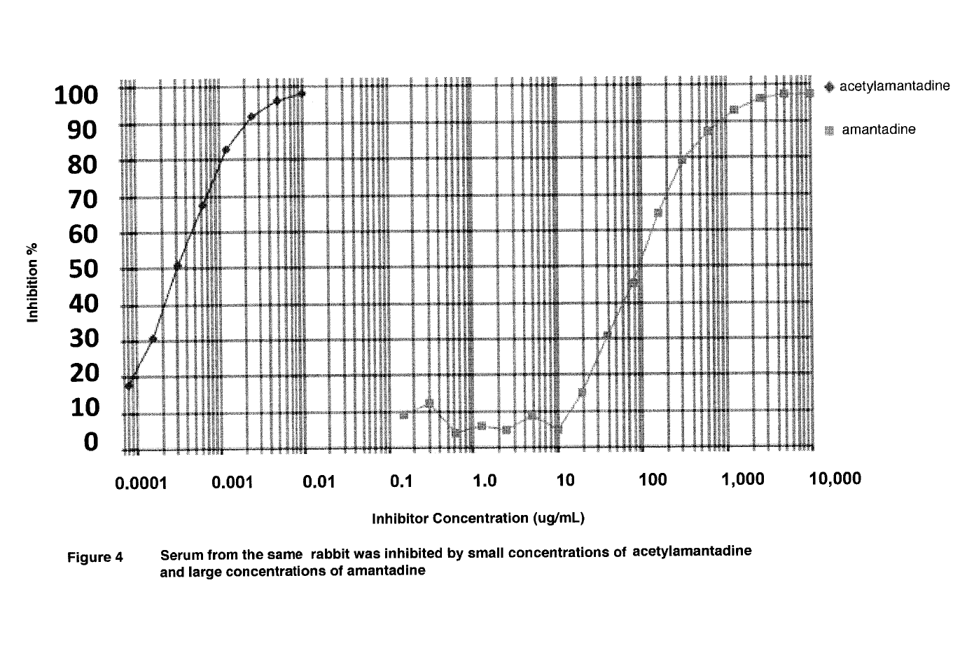

The sera from this individual rabbit also worked in this immunological assay that quantified acetyl amantadine as shown in FIG. 4. There was significant inhibition (50%) of the binding in the ELISA with ˜300pg/ mL of soluble acetylamantadine added to the dilution of serum as shown in FIG. 2. In contrast, there was significant inhibition (50%) with 100 μg/mL of amantadine as shown in FIG. 2.

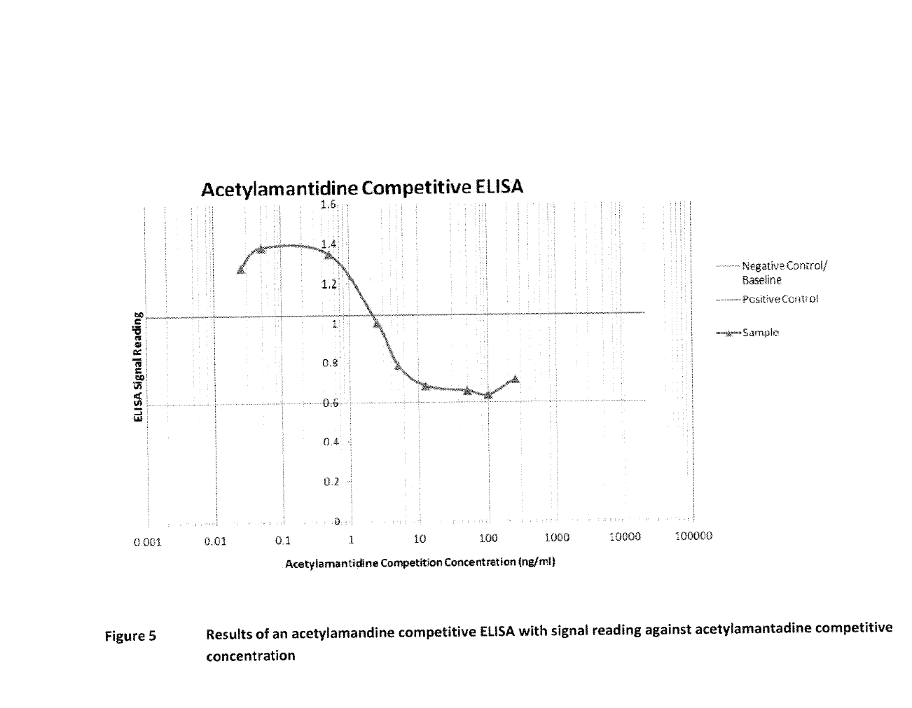

It will accordingly be understood by a person skilled in the art that the immunological assay disclosed herein may be used to detect or quantify acetylamantadine in a patient who has taken a small dose of amantadine. This is shown in FIG. 5 which illustrates the results of an acetylamandine competitive ELISA with signal reading against acetylamantadine competitive concentration. The ELISA plate coating was 2 ug/ml streptavidin and 6 ng/ml biotinylated acetylamantadine. The competition ELISA using serum at 1:200 dilution against acetylamantadine ranging from 0.025 ng/ml to 250 ng/ml. Complete inhibition inhibition occurs near 100 ng/ml of soluble acetylamantadine. The detection limit (sensitivity) is near 0.5 ng/ml to 1.0 ng/ml The quantification of acetylamantadine can be used to quantify SSAT activity and elevated SSAT activity is an indication of diseases including, but not limited, to inflammations and cancers.

It will still further be understood by a person skilled in the art that many of the details provided above are by way of example only, and are not intended to limit the scope of the invention which is to be determined with reference to the following claims.

1. A method of detecting acetylamantadine in bodily fluids of a mammal, the method comprising:
dosing the mammal with amantadine;
taking a bodily fluid sample from the mammal;
incubating the bodily fluid sample with a known concentration of antibodies specific for acetylamantadine; and
detecting acetylamantadine in the bodily fluid sample based on the inhibition of the binding of anti-acetylamantadine antibodies to acetylamantadine bound on a substrate.

2. A method of quantifying acetylamantadine in bodily fluids of a mammal, the method comprising:
dosing the mammal with amantadine;
taking a bodily fluid sample from the mammal;
incubating the bodily fluid sample with a known concentration of antibodies specific for acetylamantadine; and
quantifying acetylamantadine in the bodily fluid sample based on the inhibition of the binding of anti-acetylamantadine antibodies to acetylamantadine bound on a substrate.
